(19) United States
(12) Patent Application Publication
(10) Pub. No.: US 2021/0403859 A1
(43) Pub. Date: Dec. 30, 2021

(54) A GROWTH MEDIA COMPOSITION AND IMPROVED METHODS OF PRODUCING BIOMASS AND VALUE ADDED PRODUCT

(71) Applicant: STRING BIO PRIVATE LIMITED, Bangalore (IN)

(72) Inventors: Basavaraj PALABHANVI, Bangalore (IN); Sandeep Kumar C, Bangalore (IN); Hamsini SUBRAMANIAN, Bangalore (IN); Naga Sairam S, Bangalore (IN); Purvesh SHINGALA, Bangalore (IN); Ezhil SUBBIAN, Bangalore (IN)

(21) Appl. No.: 17/292,234

(22) PCT Filed: Nov. 11, 2019
(86) PCT No.: PCT/IB2019/059664

§ 371 Date: May 7, 2021

(30) Foreign Application Priority Data
Nov. 9, 2018 (IN) 201841042227

(51) Int. Cl. C12N 1/20

(52) U.S. Cl. CPC C12N 1/20

(57) ABSTRACT

The present disclosure relates to growth media composition. The disclosure further relates to a method of producing the biomass at higher concentration by employing a growth media composition. The disclosure further relates to a method of producing value added product employing growth media composition. The growth media composition of the present disclosure is homogenous in nature and is self-sterilized. The present disclosure provides for enhanced productivity of the biomass and the value added products, respectively employing gaseous substrate.

### TECHNICAL FIELD

The present disclosure relates to a composition, particularly to a growth media composition and to a process of preparing the growth media. The disclosure further relates to a method of producing biomass at higher concentration by employing gaseous substrate such as C1 substrates in the presence of the said growth media composition. The disclosure further relates to a method of producing value added product employing the said growth media composition, wherein the said value added products are produced at enhanced rate.

### BACKGROUND OF THE DISCLOSURE

To date, largely non-homogenous growth media was used in fermentation of C1 substrates. However, non-homogenous growth media are very challenging to scale and limit robust operation. Also, non-homogenous growth media limits the production of the biomass at higher concentration and thereby affects the yield of the fermentation products. Further, the said growth media is subjected to thermal sterilization before use in the bioreactor, which adds to additional cost to the fermentation process.

Therefore, there is a need for a growth media which is homogenous and does not need thermal sterilization which can increase the biomass concentration during fermentation and thereby causing increased fermentation products.

The object of the present disclosure is to provide a growth media composition which is homogenous in nature and is self-sterilized and which enhances the biomass concentration during fermentation. The present disclosure further elaborates a method of producing value added products using the defined growth media composition.

### SUMMARY OF THE DISCLOSURE

Accordingly, the present disclosure relates to a growth media composition comprising MgSO₄.7H₂O, CaCl₂.2H₂O, Fe,Na-EDTA, NaMoO4.2H₂O, FeSO₄.7H₂O, ZnSO₄.7H₂O, H₃BO₃, COCl₂.6H₂O, Na₂-EDTA Dihydrate, MnCl₂.4H₂O, NiCl2.6H₂O, CuSO₄.5H₂O, HNO₃, H₂SO₄/KHSO₄, H₃PO₄, KH₂PO₄, optionally along with additional nitrogen containing source phosphate containing source and sulphate containing source.

The said growth media composition is homogenous in nature and it is self-sterilized, which do not need thermal sterilization and can be directly used in the bioreactor without external sterilization.

The present disclosure further relates to a process of preparing the said growth media composition comprising mixing the components selected from a group comprising MgSO4.7H₂O, CaCl₂.2H₂O, Fe,Na-EDTA, NaMoO₄.2H₂O, FeSO₄.7H₂O, ZnSO₄.7H₂O, H₃BO₃, CoCl₂.6H₂O, Na₂-EDTA Dihydrate, MnCl₂.4H₂O, NiCl₂.6H₂O, CuSO₄.5H₂O, HNO₃, H₂SO₄/KHSO₄, H₃PO₄/KH₂PO₄, optionally along with additional nitrogen containing source, phosphate containing source, sulphate containing source or a combination thereof in a predetermined manner to obtain a homogenous growth media.

The present disclosure further relates to a method of producing biomass, comprising—culturing microorganism in the said growth media composition; and harvesting the biomass.

The present disclosure furthermore relates to a method of producing value added products, comprising—culturing microorganism in the said growth media composition; harvesting the biomass; and separating therefrom value added products produced from the said microorganism.

### DETAILED DESCRIPTION

The present disclosure relates to a composition, particularly to a growth media composition.

In an embodiment of the present disclosure, the growth media composition comprises micro element and trace element, optionally along with additional nitrogen containing source, phosphate containing source and sulphate containing source.

In another embodiment of the present disclosure, the growth media composition comprises micro element and trace element.

In another embodiment of the present disclosure, the growth media composition comprises micro element, trace element, nitrogen containing source, phosphate containing source and sulphate containing source.

In an embodiment of the present disclosure, the microelement is selected from a group comprising MgSO₄.7H₂O, CaCl₂.2H₂O and a combination thereof.

In an embodiment of the present disclosure, the trace element is selected from a group comprising FeNa-EDTA, NaMoO₄.2H₂O, FeSO₄.7H₂O, ZnSO₄.7H₂O, H₃BO₃, CoCl₂.6H₂O, Na₂-EDTA Dihydrate, MnCl₂.4H₂O, NiCl₂.6H₂O, CuSO₄.5H₂O and a combination thereof.

In an embodiment of the present disclosure, the growth media comprises MgSO₄.7H₂O, CaCl₂.2H₂O, FeNa-EDTA, NaMoO₄.2H₂O, FeSO₄.7H₂O, ZnSO₄.7H₂O, H₃BO₃, COCl₂.6H₂O, Na₂-EDTA Dihydrate, MnCl₂.4H₂O, NiCl₂.6H₂O, CuSO₄.5H₂O, HNO₃, H₂SO₄/KHSO₄, H₃PO₄/KH₂PO₄, optionally along with additional nitrogen containing source, phosphate containing source, sulphate containing source or a combination thereof.

In an embodiment of the present disclosure, the MgSO₄.7H₂O in the growth media composition is in an amount ranging from about 0.1% to 1.2%.

In another embodiment of the present disclosure, the MgSO₄.7H₂O in the growth media composition is in an amount of about 0.1%, about 0.2%, about 0.3%, about 0.4%. about 0.5%, about 0.6%, about 0.7%, about 0.8%, about 0.9%, about 1.0%, about 1.1% or about 1.2%.

In an embodiment of the present disclosure, the CaCl₂.2H₂O in the growth media composition is in an amount ranging from about 0.02% to 0.3%.

In another embodiment of the present disclosure, the CaCl₂.2H₂O in the growth media composition is in an amount of about 0.02%, about 0.04%, about 0.06%, about 0.08%. about 0.1%, about 0.12%, about 0.14%, about 0.16%, about 0.18%, about 0.20%, about 0.22%, about 0.24%, about 0.26%, about 0.28% or about 0.3%.

In an embodiment of the present, the FeNa-EDTA in the growth media composition is in an amount ranging from about 0% to 0.013%.

In an embodiment of the present, the NaMoO₄.2H₂O in the growth media composition is in an amount ranging from about 0.000026% to 0.0003%.

In an embodiment of the present, the FeSO₄.7H₂O in the growth media composition is in an amount ranging from about 0.00005% to 0.006%.

In an embodiment of the present, the ZnSO₄.7H₂O in the growth media composition is in an amount ranging from about 0.00004% to 0.0005%.

In an embodiment of the present, the H₃BO₃ in the growth media composition is in an amount ranging from about 0 ppm to 0.3 ppm.

In an embodiment of the present, the CoCl₂.6H₂O in the growth media composition is in an amount ranging from about 0.05 ppm to 0.45 ppm.

In an embodiment of the present, the Na2-EDTA Dihydrate in the growth media composition is in an amount ranging from about 0 ppm to 8 ppm.

In an embodiment of the present, the MnCl₂.4H2O in the growth media composition is in an amount ranging from about 0.02 ppm to 0.15 ppm.

In an embodiment of the present, the NiCl2.6H2O in the growth media composition is in an amount ranging from about 0.01 ppm to 0.075 ppm.

In an embodiment of the present, the CuSO4.5H₂O in the growth media composition is in an amount ranging from about 1 ppm to 50 ppm.

In an embodiment of the present disclosure, the nitrogen containing source is selected from a group comprising sodium nitrate, sodium nitrite, potassium nitrate, potassium nitrite, ammonia, ammonium hydroxide, ammonium chloride, ammonium acetate, ammonium sulphate, nitric acid, di ammonium phosphate (DAP) and any combinations thereof.

In an embodiment of the present disclosure, the phosphate containing source is selected from a group comprising potassium dihydrogen phosphate, dipotassium hydrogen phosphate, sodium dihydrogen phosphate, disodium hydrogen phosphate, phosphoric acid, di ammonium phosphate and any combinations thereof.

In an embodiment of the present disclosure, the sulphate containing source is selected from a group comprising copper sulphate, zinc sulphate, iron sulphate, magnesium sulphate, manganous sulphate, sulfuric acid and any combinations thereof.

In an embodiment of the present disclosure, in the growth media composition, ratio of elemental nitrogen to phosphate is ranging from about 1:2 to 10:1.

In another embodiment of the present disclosure, in the growth media composition, ratio of nitrogen to phosphate is about 1:2, about 1:1, about 2:1, about 3:1, about 4:1, about 5:1, about 6:1, about 7:1, about 8:1, about 9:1 or about 10:1.

In an embodiment of the present disclosure, in the growth media composition, ratio of elemental nitrogen to sulphate is ranging from about 1:1 to 10:1.

In another embodiment of the present disclosure, in the growth media composition, ratio of elemental nitrogen to sulphate is about 1:1, about 2:1, about 3:1, about 4:1, about 5:1, about 6:1, about 7:1, about 8:1, about 9:1 or about 10:1.

In an embodiment of the present disclosure, in the growth media composition, ratio of the elemental nitrogen to phosphate and the ratio of elemental nitrogen to sulphate varies depending on the microorganism cultured in the said growth media composition.

In an embodiment of the present disclosure, the growth media composition is self-sterilized by the components employed therein. The said growth media composition does not require thermal sterilization or any kind of external sterilization.

In another embodiment of the present disclosure, the growth media composition is self-sterilized by the components present in the composition, which are acidic or basic in nature. The nitrogen containing source selected from a group comprising HNO3, NH3 and a combination thereof, phosphate containing source selected from a group comprising H3PO4, KH2PO4 and a combination thereof) and sulphate containing source H2SO4 KHSO4) and a combination thereof in the growth media composition enable the composition to be self-sterilized. Further, presence of HNO3, H2SO4/KHSO4 and H3PO4/KH2PO4 in the growth media composition makes the media homogenous and brings the pH less than 3, preferably pH of about 2.5, wherein the contaminants cannot survive. So that the external sterilization requirement before using the composition is overcome.

In an embodiment of the present disclosure, the growth media composition is uniquely designed by synergistically combining HNO3, H2SO4/KHSO4 and H3PO4/KH2PO4 as nitrogen source, sulphate source and phosphate source, respectively. These components play a critical role in enabling a homogenous mixture and at the same time provide cellular nutrients in optimal ratios so as to enable high cell densities and growth rates of the biomass. In addition, the presence of acidic components in the growth media composition eliminates the need for growth media composition sterilization and negates the growth of contaminants. Thus, growth media composition of the present disclosure addresses limitations in robust process scale up with distinct advantages.

In an embodiment of the present disclosure, the growth media composition is a homogenous mixture.

In another embodiment present disclosure, the growth media composition is homogenous despite having about 10 times higher concentration in a solution and does not form any precipitate which is plays a role in obtaining high cell densities and growth rates of the biomass.

In an embodiment of the present disclosure, the growth media composition enhances the biomass productivity during fermentation.

In another embodiment of the present disclosure, the growth media enhances the biomass productivity during fermentation of gaseous substrates.

In an embodiment of the present disclosure, the growth media composition enhances the biomass productivity by at least about 2 g/l to 8 g/l of the reactor working volume per hour during fermentation of the gaseous substrates when compared to fermentation process known in the art with Continuous stirred-tank reactor (CSTR) which is about 1.8 g/l of reactor working volume per hour.

In an embodiment of the present disclosure, the growth media composition enhances the biomass productivity by at least about 2 g/l, about 3 g/l, about 4 g/l, about 5 g/l, about 6 g/l, about 7 g/l or about 8 g/l of the reactor working volume per hour during fermentation of the gaseous substrates when compared to fermentation process known in the art with Continuous stirred-tank reactor (CSTR) which is about 1.8 g/l of reactor working volume per hour.

In an embodiment of the present disclosure, the growth media composition enhances the biomass productivity by at least about 2 g/l to 8 g/l of the media per hour in fed-batch operation, semi-continuous operation and continuous mode of operation.

The present disclosure further relates to process of preparing the growth media composition.

In an embodiment of the present disclosure, the process of preparing the growth media composition comprises mixing micro element and trace element, optionally along with nitrogen containing source, phosphate containing source and sulphate containing source by a predetermined technique, at a predetermined temperature for a predetermined duration to obtain the growth media composition.

In another embodiment of the present disclosure, the process of preparing the growth media composition comprises mixing micro element and trace element by a predetermined technique, at a predetermined temperature for a predetermined duration to obtain the growth media composition.

In another embodiment of the present disclosure, the process of preparing the growth media composition comprises mixing micro element, trace element, nitrogen containing source, phosphate containing source and sulphate containing source by a predetermined technique, at a predetermined temperature for a predetermined duration to obtain the growth media composition.

In another embodiment of the present disclosure, the process of preparing the growth media composition comprises mixing MgSO4.7H2O, CaCl2.2H2O, Fe,Na-EDTA, NaMoO4.2H2O, FeSO4.7H2O, ZnSO4.7H2O, H3BO3, CoCl2.6H2O, Na2-EDTA Dihydrate, MnCl2.4H2O, NiCl2.6H2O, CuSO4.5H2O, HNO₃, H2SO4/KHSO4, H3PO4/KH2PO4 and additional nitrogen containing source, additional phosphate containing source and additional sulphate containing source, in a predetermined technique, at a predetermined temperature for a predetermined duration to obtain the growth media composition.

In another embodiment of the present disclosure, in the process of preparing the growth media composition the specified/predetermined amount of MgSO4.7H2O, CaCl2.2H2O, Fe,Na-EDTA, NaMoO4.2H2O, FeSO4.7H2O, ZnSO4.7H2O, H3BO3, CoCl2.6H2O, Na2-EDTA Dihydrate, MnCl2.4H2O, NiCl2.6H2O and CuSO4.5H2O are mixed, followed by adding water and mixed again. The predetermined amount of HNO3, H2SO4/KHSO4 and/or H3PO4/KH2PO4 are then added to the mixture and thoroughly mixed in order to cause dissolution of all the components. Thereafter, additional nitrogen containing source, additional phosphate containing source and/or additional sulphate containing source is added and stirred for about 10 minutes to obtain a homogenous solution. The final volume of the media is made up using water.

The present disclosure further relates to a method of producing biomass employing the above defined growth media composition.

In an embodiment of the present disclosure, the method of producing the biomass employing the said growth media composition causes increase in the biomass concentration during fermentation.

In another embodiment of the present disclosure, the method of producing the biomass employing the said growth media composition is concerned with increasing the biomass concentration during fermentation of gaseous substrates.

In an embodiment of the present disclosure, the method of producing the biomass comprises—
culturing microorganism in a growth media composition;
supplementing the culture with a growth media composition having acidic pH; and
harvesting the biomass to obtain the biomass.

In an embodiment of the present disclosure, in the method of producing the biomass, culturing the microorganism comprises—
inoculating the growth media composition with the microorganism, followed by providing gaseous substrate and oxygen; and
supplementing with the growth media composition.

In another embodiment of the present disclosure, in the method of producing the biomass, culturing the microorganism comprises—

inoculating the growth media composition with the microorganism, followed by providing gaseous substrate and oxygen; and

monitoring cell density of the microorganism and supplementing with the growth media composition.

In an embodiment of the present disclosure, in the method of producing the biomass, the growth media composition is having pH of less than 3, preferably pH of about 2.5. the said pH of the growth media composition enables the media composition to keep the unwanted microorganisms at bay.

In an embodiment of the present disclosure, the growth media composition supplemented during the method of producing the biomass, comprises varied components at varied amounts, which are within the ambit of the components and their amounts described above for the growth media composition.

In another embodiment of the present disclosure, in the method of producing the biomass, the growth media composition for culturing and supplement is same or different and wherein the growth media composition for supplementing the culture is having pH of less than 3, preferably pH of about 2.5.

In an embodiment of the present disclosure, in the method of producing the biomass, the culturing of the microorganism is carried out at temperature ranging from about 5° C. to 50° C. and at a pressure ranging from about 0 bar to 5 bar.

In an embodiment of the present disclosure, in the method of producing the biomass, the culturing of the microorganism is carried out at temperature of about 5° C., about 10° C., about 15° C., about 20° C., about 25° C., about 30° C., about 35° C., about 40° C., about 45° C. or about 50° C., and at a pressure of about 0 bar, about 1 bar, about 2 bar, about 3 bar, about 4 bar or about 5 bar.

In an embodiment of the present disclosure, during the method of producing the biomass, the growth media composition is supplemented once the cell density of the microorganism is ranging from about 0.15% to 2%.

In another embodiment of the present disclosure, during the method of producing the biomass, the growth media composition is supplement once the cell density of the microorganism is about 0.15%, about 0.25%, about 0.5%, about 0.75%, about 1%, about 1.25%, about 1.5%, about 1.75% or about 2%.

In an embodiment of the present disclosure, in the method of producing the biomass, the culturing of microorganism is carried out for a period ranging from about 120 hours to 900 hours.

In another embodiment of the present disclosure, in the method of producing the biomass, the culturing of microorganism is carried out for a period of about 120 hours, about 140 hours, about 160 hours, about 180 hours, about 200 hours, about 220 hours, about 240 hours, about 260 hours, about 280 hours, about 300 hours, about 320 hours, about 340 hours, about 360 hours, about 380 hours, about 400 hours, about 420 hours, about 440 hours, about 460 hours, about 480 hours, about 500 hours, about 520 hours, about 540 hours, about 560 hours, about 580 hours, about 600 hours, about 620 hours, about 640 hours, about 660 hours, about 680 hours, about 700 hours, about 720 hours, about 740 hours, about 760 hours, about 780 hours, about 800 hours, about 820 hours, about 840 hours, about 860 hours, about 880 hours or about 900 hours.

In an embodiment of the present disclosure, the method of producing biomass, culturing of the microorganism involves fermentation of gaseous substrate by microorganisms in presence of the growth media composition defined above and air.

In an embodiment of the present disclosure, the gaseous substrate is selected from a group comprising methane, natural gas, syngas, landfill gas, carbon monoxide, biogas and any combinations thereof.

In another embodiment of the present disclosure, the gaseous substrate is C1 substrate selected from a group comprising methane, methanol, carbon dioxide, carbon monoxide and any combinations thereof.

In an embodiment of the present disclosure, the gaseous substrate is at a concentration ranging from about 1 mg/L to 8 mg/L.

In another embodiment of the present disclosure, the gaseous substrate is at a concentration of about 1 mg/L, about 2 mg/L, about 3 mg/L, about 4 mg/L, about 5 mg/L, about 6 mg/L, about 7 mg/L or about 8 mg/L.

In an embodiment of the present disclosure, the gaseous substrate, such as methane is having purity ranging from about 40% to 100% is used in fermentation.

In another embodiment of the present disclosure, the methane is having a purity of about 40%, about 45%, about 50%, about 55%, about 60%, about 65%, about 70%, about 75%, about 80%, about 85%, about 90%, about 95% or about 100%.

In an embodiment of the present disclosure, the oxygen employed in the method of producing the biomass is at a concentration ranging from about 1 mg/L to 10 mg/L and having a purity ranging from about 40% to 99.9%.

In another embodiment of the present disclosure, the oxygen employed in the method of producing the biomass is at a concentration of about 1 mg/L, about 2 mg/L, about 3 mg/L, about 4 mg/L, about 5 mg/L, about 6 mg/L, about 7 mg/L, about 8 mg/L, about 9 mg/L or about 10 mg/L and the oxygen is having a purity of about 40%, about 45%, about 50%, about 55%, about 60%, about 65%, about 70%, about 75%, about 80%, about 85%, about 90%, about 95% or about 99.9%.

In an embodiment of the present disclosure, the microorganism employed in the method of producing the biomass is selected from a group comprising Methylococcus capsulatus, Methylobacterium extorquens, Methylomicrobium album, Methylocapsa acidiphila, Methylobacterium organophilum, Methylobacterium mesophilicum, Methylobacterium dichloromethanicum, Methylocella silvestris, Methylosinus trichosporium, Methylobacillus flagellatus KT, Methylibium petroleiphilum PM1, Methylobacterium nodulans, Methylobacterium populi, Methylobacterium chloromethanicum, Methylacidiphilum infernorum V4, Methylophilus methylotrophus, Methylomonas methanica, Methylobacterium rhodesianum MB 126, Methylobacter tundripaludum, Methylobacterium sp. 4-46, Methylovorus glucosetrophus SIPS-4, Mycobacterium smegmatis, Methylobacterium rhodesianum, Methylosinus sporium, Methylocella palustris, Methylobacterium fujisawaense, Methylocystis parvus, Methylovulum miyakonense, Methylobacterium rhodinum, Methylocystis echinoides, Methylomonas rubra, Methylococcus thermophilus, Methylobacterium aminovorans, Methylobacterium thiocyanatum, Methylobacterium zatmanii, Acidithiobacillus ferrivorans, Methylobacterium aquaticum, Methylobacterium suomiense, Methylobacterium adhaesivum, Methylobacterium podarium, Methylobacter whittenburyi, Crenothrix polyspora, Clonothrix fusca, Methylobacter bovis, Methylomonas aurantiaca, Methylomonas fodinarum, Methylobacterium variabile, Methylocystis minimus, Methylobacter vinelandii, Methylobacterium hispanicum, Methylomicrobium japanense, Methylococcaceae bacterium, Methylocystis methanolicus and any combination thereof.

In an embodiment of the present disclosure, in the method of producing the biomass, the pH is maintained by the basic compound or acidic compound of the nitrogen containing source, the phosphate containing source, the sulphate containing source or any combinations thereof of the growth media composition.

In another embodiment of the present disclosure, in the method of producing the biomass, the pH is maintained by the basic compound or acidic compound of the nitrogen containing source, the phosphate containing source, the sulphate containing source or any combinations thereof of the growth media composition and along with compounds selected from a group comprising sodium hydroxide, hydrochloride acid, and a combination thereof.

In an embodiment of the present disclosure, the method of producing the biomass comprises substrate dependent control strategy, which simplifies the manner of performing the method and reduces the cost involved in performing the method, thus making the said method significantly efficient and economical in producing the biomass at enhanced rate.

In an embodiment of the present disclosure, in the method of producing the biomass, the substrate dependent control strategy comprises controlling or maintaining the pH by the basic or the acidic compound of the nitrogen containing source, the phosphate containing source, the sulphate containing or any combinations thereof of the growth media composition.

In an embodiment present disclosure, the said method of producing the biomass causes enhanced production of the steady state biomass density ranging from about 2% to 5%, wherein enhanced productivity is ranging from about 0.5 g L⁻¹ hour⁻¹ to 8 g L⁻¹ hour⁻¹.

In an embodiment of the present disclosure, in the said method of producing the biomass, enhanced biomass is produced by controlling mass flow rate of carbon content up to about 30 grams per liter of reactor working volume per hour, controlling oxygen content up to about 130 grams per liter of reactor working volume per hour, controlling the nitrogen content up to about 24 grams per liter of reactor working volume per day, controlling the phosphate content up to about 14 grams per liter of reactor working volume per day, controlling the sulphate content up to about 16 grams per liter of reactor working volume per day, controlling the micro elements content up to about 50 grams per liter of reactor working volume per day and controlling the trace elements content up to about 1 gram per liter of reactor working volume per day.

In an embodiment of the present disclosure, the said method of producing the biomass employs the growth media composition defined above which is self-sterilized and does not require thermal sterilization or any kind of external sterilization means. Thus, the said method of producing the biomass is significantly economical and energetically superior.

In an embodiment of the present disclosure, in the method of producing the biomass, an important aspect lies in the fact that the culturing of the microorganisms, in presence of the gaseous substrate to obtain the biomass, is taking place in presence of supplemental/continuous media having components that allow storage of media at pH of less than 3, preferably pH of about 2.5, thereby regulating the growth of contaminants. Although the media composition of the present disclosure is prepared to achieve this result, a person skilled in the art would understand that any media composition that achieves this requirement of acidic pH of less than 3, preferably pH of about 2.5 can be alternatively employed to achieve the same.

The present disclosure further relates to a method of producing value added products employing the growth media composition.

In an embodiment of the present disclosure, the method of producing value added products, comprises—
culturing microorganism in a growth media composition;
supplementing the culture with a growth media composition having acidic pH; harvesting biomass; and
separating therefrom value added product produced from the biomass.

In another embodiment of the present disclosure, in the method of producing value added products, culturing the microorganism comprises—
inoculating the growth media with the microorganism, followed by providing gaseous substrate and oxygen; and
supplementing with the growth media.

In another embodiment of the present disclosure, in the method of producing value added product, culturing the microorganism comprises—
inoculating the growth media with the microorganism, followed by providing gaseous substrate and oxygen; and
monitoring cell density of the microorganism and supplementing with the growth media.

In an embodiment of the present disclosure, in the method of producing the biomass, the growth media composition is having pH of less than 3, preferably pH of about 2.5. The said pH of the growth media composition enables the media composition to keep the unwanted microorganisms at bay.

In an embodiment of the present disclosure, the growth media composition supplemented during the method of producing the biomass, comprises varied components at varied amounts, which are within the ambit of the components and their amounts described above for the growth media composition.

In another embodiment of the present disclosure, in the method of producing the value added product, the growth media composition for culturing and supplementing is same or different; and wherein the growth media composition for supplementing the culture is having pH of less than 3, preferably pH of about 2.5.

In an embodiment of the present disclosure, the method of producing value added products, culturing of the microorganism involves fermentation of gaseous substrate by microorganisms in presence of the growth media composition defined above and air.

In an embodiment of the present disclosure, the method of producing value added products further comprises separation and purification of the value added products.

In an embodiment of the present disclosure, the method produces bio-based value added products from gaseous substrates effectively, wherein said process can be carried out in a centralized facility with a large scale fermentation or in a decentralized facility with smaller scales of fermentation.

In an embodiment of the present disclosure, the microorganism employed in the method of producing the value added product can be wild type microorganism, recombinant microorganism or a combination thereof.

In an embodiment of the present disclosure, in the method of producing value added product, the growth media composition is supplemented once the cell density of the microorganism is ranging from about 0.15% to 2%.

In another embodiment of the present disclosure, in the method of producing value added product, the growth media composition is supplemented once the cell density of the microorganism is about 0.15%, about 0.25%, about 0.5%, about 0.75%, about 1%, about 1.25%, about 1.5%, about 1.75% or about 2%.

In an embodiment of the present disclosure, in the method of producing value added product, the culturing of the microorganism is carried out at a pressure ranging from about 0 bar to 5 bar.

In an embodiment of the present disclosure, in the method of producing value added product the culturing of the microorganism is carried out at a pressure of about 0 bar, about 1 bar, about 2 bar, about 3 bar, about 4 bar or about 5 bar.

In an embodiment of the present disclosure, in the method of producing value added product, the culturing of the microorganism is carried out at a temperature ranging from about 20 to 50° C.

In another embodiment of the present disclosure, in the method of producing value added product, the culturing of the microorganism is carried out at a temperature of about 20° C., about 25° C., about 30° C., about 35° C., about 40° C., about 45° C. or about 50° C.

In an embodiment of the present disclosure, in the method of producing value added product, the culturing of the microorganism is carried out for a period ranging from about 120 hours to 900 hours.

In an embodiment of the present disclosure, in the method of producing value added product, the culturing of the microorganism is carried out for a period of about 120 hours, about 140 hours, about 160 hours, about 180 hours, about 200 hours, about 220 hours, about 240 hours, about 260 hours, about 280 hours, about 300 hours, about 320 hours, about 340 hours, about 360 hours, about 380 hours, about 400 hours, about 420 hours, about 440 hours, about 460 hours, about 480 hours, about 500 hours, about 520 hours, about 540 hours, about 560 hours, about 580 hours, about 600 hours, about 620 hours, about 640 hours, about 660 hours, about 680 hours, about 700 hours, about 720 hours, about 740 hours, about 760 hours, about 780 hours, about 800 hours, about 820 hours, about 840 hours, about 860 hours, about 880 hours or about 900 hours

In an embodiment of the present disclosure, in the method of producing value added product, the culturing of microorganism in the growth media composition causes fermentation of gaseous substrate.

In an embodiment of the present disclosure, in the method of producing the value added product, the gaseous substrate is selected from a group comprising methane, natural gas, syngas, landfill gas, carbon monoxide, biogas and any combinations thereof.

In another embodiment of the present disclosure, the gaseous substrate is C1 substrate selected from a group comprising methane, methanol, carbon dioxide, carbon monoxide and any combination thereof.

In an embodiment of the present disclosure, in the method of producing the value added product the gaseous substrate is at a concentration ranging from about 1 mg/L to 8 mg/L.

In another embodiment of the present disclosure, in the method of producing the value added product the gaseous substrate is at a concentration of about 1 mg/L, 2 mg/L, 3 mg/L, 4 mg/L, 5 mg/L, 6 mg/L, 7 mg/L or 8 mg/L.

In another embodiment of the present disclosure, in the method of producing value added product, the methane is having a purity of about 40%, about 45%, about 50%, about 55%, about 60%, about 65%, about 70%, about 75%, about 80%, about 85%, about 90%, about 95% or about 100%.

In an embodiment of the present disclosure, in the method of producing value added product, the oxygen is at a concentration ranging from about 1 mg/L to 10 mg/L and having a purity ranging from about 40% to 99.9%.

In another embodiment of the present disclosure, in the method of producing value added product, the oxygen is at a concentration of about 1 mg/L about 2 mg/L, about 3 mg/L, about 4 mg/L, about 5 mg/L, about 6 mg/L, about 7 mg/L, about 8 mg/L, about 9 mg/L or about 10 mg/L and the oxygen is having a purity of about 40%, about 45%, about 50%, about 55%, about 60%, about 65%, about 70%, about 75%, about 80%, about 85%, about 90%, about 95% or about 99.9%.

In the embodiment of the present disclosure, in the method of producing value added product, the fermenter and parameters are optimized with an objective that the gas (C1 carbon) conversion rate is at least 1 g/L/h. The gas conversion rate refers to the rate of utilization of the gaseous substrate that is fed to the reactor.

In another embodiment in the method of producing value added product, the gas (C1 carbon) conversion rate is ranging from about 0.1 g/L/h to 20 g/L/h.

In another embodiment of the present disclosure in the method of producing value added product, the gas (C1 carbon) conversion rate is about 0.1 g/L/h, about 0.5 g/L/h, about 1.0 g/L/h, about 2 g/L/h, about 3 g/L/h about 4 g/L/h about 5 g/L/h about 6 g/L/h about 7 g/L/h about 8 g/L/h about 9 g/L/h about 10 g/L/h, 11 g/L/h, about 12 g/L/h, about 13 g/L/h about 14 g/L/h about 15 g/L/h about 16 g/L/h about 17 g/L/h about 18 g/L/h about 19 g/L/h about 20 g/L/h.

In an embodiment of the present disclosure, in the method of producing value added product, during culturing of microorganism, process parameters are optimized for ideal fermentation of gaseous substrates. The process parameters that are optimized include but not limiting to gaseous substrate flow rate, air flow rate (oxygen), ratio of gaseous substrate to air, superficial gas velocity, rate of gaseous substrate recycle, dissolved carbon-di-oxide concentration in the media, gas liquid separation, specific growth rate, fermentation temperature, agitation, pressure and pH or any combination of parameters.

In another embodiment of the present disclosure, the key parameters that are optimized during fermentation are gas residence time, gas transfer coefficient, gaseous substrate flow rate/methane flow rate, air flow rate, ratio of gaseous substrate: air or ratio of methane: air and pressure or any combination of parameters thereof.

In an embodiment of the present disclosure, in the method of producing value added product, the gaseous flow rate refer to methane or combination of methane and air (oxygen) flow rate that further relates to volume of gas fed to the fermentation medium per unit of time. Optimum gas feed rates vary depending on the size of the fermenter. The gas flow rate varies from about 0.05 vvm to 3 vvm.

In an embodiment of the present disclosure, in the method of producing value added product, the gaseous substrate, such as methane is fed into the growth media composition defined above, wherein the gaseous substrate may not be completely utilized and a percentage of the methane may be pushed out of the reactor unused. Hence, exit stream will have varying amounts of the gaseous substrate that is emitted together with carbon dioxide. The ratios of methane and carbon dioxide in the exit gas stream vary often with higher amounts of carbon dioxide than methane being present. This exit stream is re-cycled into the reactor to further enhance the overall yield of gaseous substrate conversion. The rate of the gaseous substrate in the recycled stream is optimized for maximum product conversion. The carbon dioxide in the exit stream from the fermenter is scrubbed and a pure gaseous stream is recycled into the fermenter. In few instances, the exit stream is recycled as is. As the gaseous substrate is being continuously introduced into the medium, the methane is also constantly separated from the liquid and pushed out of the fermenter.

In an embodiment of the present disclosure, in the method of producing value added product, air flow rate relates to volume of oxygen fed into the growth media composition per unit of time. Optimum air flow rates vary depending on the size of the reactor.

In an embodiment of the present disclosure, in the method of producing value added product, the oxygen flow rate varies depending on the size of the reactor. The workable range of oxygen flow rate varies from about 0.05 vvm to 3 vvm. In some instance, it can vary from about 0.1 vvm to 1 vvm.

In an embodiment of the present disclosure, in the method of producing value added product, ratio of gaseous substrate to oxygen is ranging from about 1:0.1 to 1:5.

In another embodiment of the present disclosure, in the method of producing value added product, ratio of gaseous substrate to oxygen is about 1:0.1, about 1:0.2, about 1:0.3, about 1:0.4, about 1:0.5, about 1:0.6, about 1:0.7, about 1:0.8, about 1:0.9, about 1:1, about 1:2, about 1:3, about 1:4 or about 1:5.

In an embodiment of the present disclosure, in the method of producing value added product, superficial gas velocity employed is ranging from about 0.01 m/s to 0.5 m/s.

In another embodiment of the present disclosure, in the method of producing value added product, superficial gas velocity employed is about 0.01 m/s, about 0.02 m/s, about 0.03 m/s, about 0.04 m/s or about 0.05 m/s.

In an embodiment of the present disclosure, in the method of producing value added product, the microorganism is selected from a group comprising Methylococcus capsulatus, Methylobacterium extorquens, Methylomicrobium album, Methylocapsa Methylobacterium organophilum, Methylobacterium mesophilicum, Methylobacterium dichloromethanicum, Methylocella silvestris, Methylosinus trichosporium, Methylobacillus flagellatus KT, Methylibium petroleiphilum PM1, Methylobacterium nodulans, Methylobacterium populi, Methylobacterium chloromethanicum, Methylacidiphilum infernorum V4, Methylophilus methylotrophus, Methylomonas methanica, Methylobacterium rhodesianum MB 126, Methylobacter tundripaludum, Methylobacterium sp. 4-46, Methylovorus glucosetrophus SIPS-4, Mycobacterium smegmatis, Methylobacterium rhodesianum, Methylosinus sporium, Methylocella palustris, Methylobacterium fujisawaense, Methylocystis parvus, Methylovulum miyakonense, Methylobacterium rhodinum, Methylocystis echinoides, Methylomonas rubra, Methylococcus thermophilus, Methylobacterium aminovorans, Methylobacterium thiocyanatum, Methylobacterium zatmanii, Acidithiobacillus ferrivorans, Methylobacterium aquaticum, Methylobacterium suomiense, Methylobacterium adhaesivum, Methylobacterium podarium, Methylobacter whittenburyi, Crenothrix polyspora, Clonothrix fusca, Methylobacter bovis, Methylomonas aurantiaca, Methylomonas fodinarum, Methylobacterium variabile, Methylocystis minimus, Methylobacter vinelandii, Methylobacterium hispanicum, Methylomicrobium japanense, Methylococcaceae bacterium, Methylocystis methanolicus and any combination thereof.

In an embodiment of the present disclosure, in the method of producing value added product, the pH is maintained by the basic compound or acidic compound of the nitrogen containing source, the phosphate containing source, the sulphate containing source or any combinations thereof of the growth media composition.

In another embodiment of the present disclosure, in the method of producing value added product, the pH is maintained by the basic component or acidic component of the nitrogen containing source, the phosphate containing source, the sulphate containing source or any combinations thereof of the growth media, along with compounds selected from a group comprising sodium hydroxide, hydrochloride acid and a combination thereof.

In another embodiment of the present disclosure, in the method of producing value added product, there is substrate dependent control strategy, which simplifies the manner of performing the method and reduces the cost involved in performing the method, thus the said method is significantly efficient and economical in producing the value added product.

In an embodiment of the present disclosure, the substrate dependent control strategy comprises controlling or maintaining the pH by the basic component or acidic component of the nitrogen containing source, the phosphate containing source, the sulphate containing source or any combinations thereof of the growth media composition.

In an embodiment of the present disclosure, the said method of producing value added product causes enhanced production of the value added products, such as lactic acid, succinic acid, formic acid, acetic acid, malic acid, beta-carotene, lutein, zeaxanthin, lycopene, astaxanthin, methanobactin annatto, peptides, ectoine, indigo and mandelic acid when compared to the methods known in the art. The enhanced rate of production of the value added products is particularly ascribed to the growth media composition of the present disclosure and to the tandem working of the growth media composition and the process parameters employed in the said method of producing the value added products.

In an embodiment of the present disclosure, the lactic acid is produced at a concentration ranging from about 5 g/l to 120 g/l, the succinic acid is produced at a concentration ranging from about 5 g/l to 50 g/l, the formic acid is produced at a concentration ranging from about 5 g/l to 50 g/l, the acetic acid is produced at a concentration ranging from about 5 g/l to 50 g/l, the malic acid is produced at a concentration ranging from about 5 g/l to 50 g/l, the beta-carotene is produced at a concentration ranging from about 0.5 g/l to 50 g/l, the lutein, the zeaxanthin, the lycopene, the peptides at a range of 0.1 to 10 g/L, the ectoine at a range of 0.1 to 10 g/L, the indigo at a range of 0.1 to 10 g/L, the mandelic acid at a range of 0.1 to 20 g/L and the annatto is produced at concentration ranging from about 0.5 g/l to 5 g/l, respectively and the methanobactin is produced at a concentration ranging from about 1 g/l to 5 g/l.

In an embodiment of the present disclosure, enhanced production of value added product in the said method is caused by controlling mass flow rate of carbon content up to about 30 grams per liter of reactor working volume per hour, controlling mass flow rate of oxygen content up to about 130 grams per liter of reactor working volume per hour, controlling mass flow rate of nitrogen content up to about 24 grams per liter of reactor working volume per day, controlling mass flow rate of phosphate up to about 14 grams per liter of reactor working volume per day, controlling mass flow rate of sulphate up to about 16 grams per liter of reactor working volume per day, controlling mass flow rate of the micro elements up to about 50 grams per liter of reactor working volume per day and controlling mass flow rate of the trace elements up to about 1 grams per liter of reactor working volume per day, wherein the said controlling of mass flow rate of the various components of the growth media composition is achieved by appropriately supplementing the reactor with the growth media composition during the method of producing the value added product.

In an embodiment of the present disclosure, the said method of producing the value added product employs the growth media composition defined above which is self-sterilized and does not require thermal sterilization or any other external sterilization technique. In general, media will be prepared by supplying micro elements, macro elements and main nutrients, and hence it acts as a good source for growth of microorganisms. Environment (water, air, etc.) contains various microorganisms which grow when they come in contact with media. Since these are unwanted microorganisms, media need to be sterilized in order to kill them (Stanbury et al., 2017). Sterilization is carried out mostly by moist heat in the form of saturated steam under pressure in the autoclave. Similarly, NMS media prepared for cultivation of methanotroph is also steam sterilized before use (Nunes J J et al., 2016). As the process scaled up, sterilization of media is a significant cost as it requires steam and power. However, growth media composition of the present disclosure does not require steam sterilization as the pH of the growth media composition is less than 3, preferably pH is about 2.5, which keeps the unwanted microorganisms at bay. Thus, the said method of producing the value added product is significantly economical and energetically superior, in addition to producing value added products at an enhanced rate.

In an embodiment of the present disclosure, in the method of producing the value added product, an important aspect lies in the fact that the culturing of the microorganisms, in presence of the gaseous substrate to obtain the value added product, is taking place in presence of supplemental/continuous media having components at acidic pH of less than 3, preferably pH of about 2.5, thereby regulating the growth of contaminants. Although the media composition of the present disclosure is prepared to achieve this result, a person skilled in the art would understand that any media composition that achieves this requirement of acidic pH of less than 3, preferably pH of about 2.5 can be alternatively employed to achieve the same.

The said method of producing the biomass and/or the method of producing the valued added product described in the present disclosure employs stirred tank reactors for high efficiency of gaseous substrate fermentation. In the prior art stirred tank reactors are not known to cause high efficiency gaseous substrate fermentation for producing enhanced biomass and/or value added products. The improved efficiency achieved in the stirred tank reactors by the methods of the present disclosure is particularly ascribed to the growth media composition of the present disclosure and to the tandem working of the growth media composition and the process parameters employed in the said methods of producing the biomass and the value added products, respectively.

The growth media composition described in the present disclosure is capable of meeting growth media requirements for higher productivity of the biomass and higher productivity of the value added products in stirred tank reactors. The design of the process parameters and the growth media composition which avoids thermal sterilization offers an economically and energetically superior process/method for producing enhanced biomass and value added products, respectively.

The method of producing the biomass and the method of producing value added products described in the present disclosure involves substrate dependent control strategies which are helpful in easy operations and reduction of cost of the operation. The use of acidic nutrients or basic nutrients in the growth media composition for maintenance of pH at set value helps in reduction of acid/base consumption during the method of producing the biomass and the method of producing the value added product, respectively. The components of the growth media composition of the present disclosure acts as both nutrient sources and pH control agents. The growth media composition with the ability to engineer the process parameter for efficient performance of the described methods in addition to producing the biomass and the value added products, respectively at an enhanced rate is the advantages of the present disclosure.

Additional embodiments and features of the present disclosure will be apparent to one of ordinary skill in art based upon description provided herein. The embodiments herein provide various features and advantageous details thereof in the description. Descriptions of well-known/conventional methods and techniques are omitted so as to not unnecessarily obscure the embodiments herein. The examples provided herein are intended merely to facilitate an understanding of ways in which the embodiments herein may be practiced and to further enable those of skill in the art to practice the embodiments herein. Accordingly, the following examples should not be construed as limiting the scope of the embodiments herein.

### EXAMPLES

### Example 1: Production of Biomass by Fermentation of Methane

About 5 liter of stirred tank reactor was filled with about 4 liter of growth media composition comprising about 0.1% of MgSO₄.7H2O, about 0.02% of CaCl2.2H2O, about 0.0004% of Fe,Na-EDTA, about 0.00003% of NaMoO4.2H2O, about 0.00005% of FeSO4.7H2O, about 0.00004% of ZnSO4.7H2O, about 15 ppb of H3BO3, about 50 ppb of CoCl2.6H2O, about 0.25 ppm of Na2-EDTA Dihydrate, about 20 ppb of MnCl2.4H2O, about 10 ppb of NiCl2.6H2O, about 1 ppm of CuSO4.5H2O, and 0.025% of nitrogen in form of nitrate. The said growth media was inoculated with a starter culture of M. capsulalus. The reactor run was initiated with sparging about 0.1 1 pm of about 99.9% pure methane and 0.05 1 pm of about 99.9% pure oxygen at the bottom of the impeller. The reactor was maintained at about one atmospheric pressure. Once the cell density in the reactor reached about 0.3% biomass, the growth media composition comprising about 0.8% of MgSO4.7H2O, about 0.2% of CaCl2.2H2O, about 0.008% of Fe,Na-EDTA, about 0.0002% of NaMoO4.2H2O, about 0.0025% of FeSO4.7H2O, about 0.0003% of ZnSO4.7H2O, about 150 ppb of H3BO3, about 300 ppb of CoCl2.6H2O, about 4 ppm of Na2-EDTA Dihydrate, about 100 ppb of MnCl2.4H2O, about 50 ppb of NiCl2.6H2O, about 15 ppm of CuSO4.5H2O , about 0.5% of HNO3 and about 0.5% of H2SO4 was added to the reactor, wherein the growth media composition is homogeneous and having pH of less than 3, preferably pH of about 2.5. The total nitrogen content of 0.5% was made by adding NaNO3 in the growth media.

Fermentation broth containing about 3.5% of solid biomass was continuously drawn out. During the reaction, pH of 6.8±0.3 was controlled by acidic and basic compounds of nitrogen containing source, phosphate sources and sulphate containing sources along with NaOH/HCl.

The method resulted in about 3.5% biomass in the reactor.

The growth media composition used for the production of biomass in this Example was as-prepared, without external sterilization. There was no contamination observed in the media tank throughout the process.

### Example 2: Production of Biomass by Fermentation of Methane using Ammonia as Nitrogen Containing Source in the Growth Media Composition

About 5 liter of stirred tank reactor was filled with about 4 liter of growth media composition comprising about 0.1% of MgSO₄.7H₂O, about 0.02% of CaCl₂.2H2O, about 0.0004% of Fe,Na-EDTA, about 0.00003% of NaMoO₄.2H₂O, about 0.00005% of FeSO₄.7H₂O, about 0.00004% of ZnSO₄.7H₂O, about 15 ppb of H₃BO₃, about 50 ppb of CoCl₂.6H₂O, about 0.25 ppm of Na₂-EDTA Dihydrate, about 20 ppb of MnCl₂.4H₂O, about 10 ppb of NiCl₂.6H₂O, about 1 ppm of CuSO₄.5H₂O, and 0.025% of nitrogen in form of liquid ammonia. The said growth media composition was inoculated with a starter culture of M. capsulalus. The reactor run was initiated with sparging about 0.1 1 pm of about 99.9% pure methane and about 0.06 1 pm of about 90% pure oxygen at the bottom of the impeller. The reactor was maintained at about one atmospheric pressure. Once the cell density in the reactor reached about 0.5% biomass, the growth media comprising about 0.4% of MgSO₄.7H₂O, about 0.1% of CaCl₂.2H₂O, about 0.0045% of Fe,Na-EDTA, about 0.0001% of NaMoO₄.2H₂O, about 0.0012% of FeSO₄.7H₂O, about 0.00015% of ZnSO₄.7H₂O, about 85 ppb of H₃BO₃, about 150 ppb of CoCl₂.6H₂O, about 2.5 ppm of Na₂-EDTA Dihydrate, about 50 ppb of MnCl₂.4H₂O, about 25 ppb of NiCl₂.6H₂O, about 7 ppm of CuSO₄.5H₂O, about 0.3% of H2SO4, and about 0.1% of H3PO4 was added to the reactor wherein the growth media composition is homogeneous and having pH of less than 3, preferably pH of about 2.5. The liquid ammonia is added to reactor separately in order make nitrogen content of media to about 0.25%.

Fermentation broth containing about 2 to 2.5% of solid biomass was continuously drawn out. During the reaction, pH was controlled by acidic and basic compounds of nitrogen containing source, phosphate sources and sulphate containing sources along with NaOH/HCl.

The method resulted in about 2.2±0.3% biomass in the reactor.

The growth media composition used for the production of biomass in this Example was as-prepared, without external sterilization.

### Example 3: Production of Biomass by Fermentation of Methane

About 10 liter of stirred tank reactor was filled with about 8 liter of growth media composition comprising about 0.1% of MgSO₄.7H₂O, about 0.02% of CaCl₂.2H₂O, about 0.0004% of Fe,Na-EDTA, about 0.00003% of NaMoO₄.2H₂O, about 0.00005% of FeSO₄.7H₂O, about 0.00004% of ZnSO₄.7H₂O, about 15 ppb of H₃BO₃, about 50 ppb of CoCl₂.6H₂O, about 0.25 ppm of Na₂-EDTA Dihydrate, about 20 ppb of MnCl₂.4H₂O, about 10 ppb of NiCl₂.6H₂O, about 1 ppm of CuSO₄.5H₂O, and 0.025% of nitrogen in form of potassium nitrate and sodium nitrate, The said growth media composition was inoculated with starter culture of M. trichosposrium. The reactor run was initiated with sparging about 0.1 1 pm of about 99.9% pure methane and about 0.11 pm of about 70% pure oxygen. As soon as total solid biomass of about 0.25% was attained in the fermentation broth, then continuous supply of the growth media was started. Growth media was comprising about 0.4% of MgSO₄.7H₂O, about 0.1% of CaCl₂.2H₂O, about 0.0045% of Fe,Na-EDTA, about 0.0001% of NaMoO₄.2H₂O, about 0.0012% of FeSO₄.7H₂O, about 0.00015% of ZnSO₄.7H₂O, about 85 ppb of H₃BO₃, about 150 ppb of CoCl₂.6H₂O, about 2.5 ppm of Na₂-EDTA Dihydrate, about 50 ppb of MnCl₂.4H₂O, about 25 ppb of NiCl₂.6H₂O, about 7 ppm of CuSO₄.5H₂O, about 0.2% of H2SO4 and about 0.08% of H3PO4 and total nitrogen content of 0.25% was made by adding NaNO3 in the growth media wherein the growth media composition is homogeneous and having pH of less than 3, preferably pH of about 2.5. Copper content of the growth media was modulated from 3 ppm to 10 ppm, where 7 ppm was found best to attain good growth of M. trichosposrium and good production of micobactin. During the reaction, the pH of the media was maintained at about 6.5±0.3 by using diluted NaOH and H₂SO₄.

The growth of M. trichosposrium was monitored by assessing the samples from the reactor about 6 hours to 8 hours. Methane and carbon dioxide in the exit gas are monitored using a biogas analyzer. The growth media composition used for the production of biomass in this Example was as-prepared, without external sterilization. There was no contamination observed in the media tank throughout the process.

### Example 4: Production of Value Added Product (Lactic Acid)

About 5 liter of stirred tank reactor was filled with about 4 liter of growth media composition comprising about 0.1% of MgSO₄.7H₂O, about 0.02% of CaCl₂.2H₂O, about 0.0004% of Fe,Na-EDTA, about 0.00003% of NaMoO₄.2H₂O, about 0.00005% of FeSO₄.7H₂O, about 0.00004% of ZnSO₄.7H₂O, about 15 ppb of H₃BO₃, about 50 ppb of CoCl₂.6H₂O, about 0.25 ppm of Na₂-EDTA Dihydrate, about 20 ppb of MnCl₂.4H₂O, about 10 ppb of NiCl₂.6H₂O, about 1 ppm of CuSO₄.5H₂O, and 0.025% of nitrogen in form of nitrate. The said growth media composition was inoculated with a starter culture of M. capsulalus which was engineered for the production of lactic acid. The reactor run was initiated with sparging about 0.1 1 pm of about 99.9% pure methane and about 0.05 1 pm of about 99.9% pure oxygen at the bottom of the impeller. The reactor was maintained at about one atmospheric pressure. Once the cell density in the reactor reached about 0.15%-1% biomass, the growth media comprising about 0.4% of MgSO₄.7H₂O, about 0.1% of CaCl₂.2H₂O, about 0.0045% of Fe,Na-EDTA, about 0.0001% of NaMoO₄.2H₂O, about 0.0012% of FeSO₄.7H₂O, about 0.00015% of ZnSO₄.7H₂O, about 85 ppb of H₃BO₃, about 150 ppb of CoCl₂.6H₂O, about 2.5 ppm of Na₂-EDTA Dihydrate, about 50 ppb of MnCl₂.4H₂O, about 25 ppb of NiCl₂.6H₂O, about 7 ppm of CuSO₄.5H₂O and about 0.25% of nitrogen in form of nitrate was added to the reactor, wherein the growth media composition is homogeneous and having pH of less than 3, preferably pH of about 2.5.

During the reaction, pH of 6.3 was controlled by acidic and basic compounds of nitrogen containing source, phosphate sources and sulphate containing sources along with NaOH/HCl.

The temperature in the reactor was controlled at about 45° C.

The method resulted in about 1±0.2% biomass in the reactor.

The lactic acid present in the media is measured on HPLC. The amount of lactic acid produced in the reaction is about 3 g/L.

### Example 5: Production of Biomass by Fermentation of Biogas

About 5 liter of stirred tank reactor was filled with about 4 liter of growth media composition comprising about 0.1% of MgSO₄.7H₂O, about 0.02% of CaCl₂.2H₂O, about 0.0004% of Fe,Na-EDTA, about 0.00003% of NaMoO₄.2H₂O, about 0.00005% of FeSO₄.7H₂O, about 0.00004% of ZnSO₄.7H₂O, about 15 ppb of H₃BO₃, about 50 ppb of CoCl₂.6H₂O, about 0.25 ppm of Na₂-EDTA Dihydrate, about 20 ppb of MnCl₂.4H₂O, about 10 ppb of NiCl₂.6H₂O, about 1 ppm of CuSO₄.5H₂O, and 0.025% of nitrogen, The said growth media composition was inoculated with a starter culture of M. capsulalus. The reactor run was initiated with sparging about 0.18 1 pm of about 60% pure biogas and about 0.09 1 pm of about 90% pure oxygen at the bottom of the impeller. The reactor was maintained at about one atmospheric pressure. Once the cell density in the reactor reached about 0.2% biomass, the growth media comprising about 0.4% of MgSO₄.7H₂O, about 0.1% of CaCl₂.2H₂O, about 0.0045% of Fe,Na-EDTA, about 0.0001% of NaMoO₄.2H₂O, about 0.0012% of FeSO₄.7H₂O, about 0.00015% of ZnSO₄.7H₂O, about 85 ppb of H₃BO₃, about 150 ppb of CoCl₂.6H₂O, about 2.5 ppm of Na₂-EDTA Dihydrate, about 50 ppb of MnCl₂.4H₂O, about 25 ppb of NiCl₂.6H₂O, about 7 ppm of CuSO₄.5H₂O and about 0.25% of nitrogen was added to the reactor, wherein the growth media composition is homogeneous and having pH of less than 3, preferably pH of about 2.5.

Fermentation broth containing about 2% to 2.5% of solid biomass was continuously drawn out.

During the reaction, pH of 6.8 was controlled by acidic and basic compounds of nitrogen containing source, phosphate sources and sulphate containing sources along with NaOH/HCI. The method resulted in about 2.2±0.3% biomass in the reactor.

The growth media composition used for the production of biomass in this Example was as-prepared, without external sterilization. There was no contamination observed in the media tank throughout the process.

### Example 6: Production of Biomass by Fermentation of Natural Gas

About 5 liter of stirred tank reactor was filled with about 4 liter of growth media composition comprising about 0.1% of MgSO₄.7H₂O, about 0.02% of CaCl₂.2H₂O, about 0.0004% of Fe,Na-EDTA, about 0.00003% of NaMoO₄.2H₂O, about 0.00005% of FeSO₄.7H₂O, about 0.00004% of ZnSO₄.7H₂O, about 15 ppb of H₃BO₃, about 50 ppb of CoCl₂.6H₂O, about 0.25 ppm of Na₂-EDTA Dihydrate, about 20 ppb of MnCl₂.4H₂O, about 10 ppb of NiCl₂.6H₂O, about 1 ppm of CuSO₄.5H₂O, and 0.025% of nitrogen. The said growth media composition was inoculated with a starter culture of M. capsulalus. The reactor run was initiated with sparging about 0.13 1 pm of natural gas comprising about 90.5% of methane, about 5.5% of ethane, about 1.75% of propane, about 0.5% of butane, about 0.25% of pentane, about 0.25% of carbon dioxide and about 1.25% of nitrogen and 0.07 1 pm of about 90% pure oxygen at the bottom of the impeller. The reactor was maintained at about one atmospheric pressure. Once the cell density in the reactor reached about 0.15-2% biomass, the growth media comprising about 0.4% of MgSO₄.7H₂O, about 0.1% of CaCl₂.2H₂O, about 0.0045% of Fe,Na-EDTA, about 0.0001% of NaMoO₄.2H₂O, about 0.0012% of FeSO₄.7H₂O, about 0.00015% of ZnSO₄.7H₂O, about 85 ppb of H₃BO₃, about 150 ppb of CoCl₂.6H₂O, about 2.5 ppm of Na₂-EDTA Dihydrate, about 50 ppb of MnCl₂.4H₂O, about 25 ppb of NiCl₂.6H₂O, about 7 ppm of CuSO₄.5H₂O, about 0.3% of HNO3, about 0.3% of H2SO4, and about 0.09% of H3PO4 was added to the reactor, wherein the growth media composition is homogeneous and having pH of less than 3, preferably pH of about 2.5. The total nitrogen content of 0.25% was made by adding NaNO3 in the growth media.

Fermentation broth containing about 2% to 2.5% of solid biomass was continuously drawn out.

During the reaction, pH was controlled by acidic and basic compounds of nitrogen containing source, phosphate sources and sulphate containing sources along with NaOH/HCl.

The method resulted in about 2.2±0.3% biomass in the reactor.

The growth media composition used for the production of biomass in this Example was as-prepared, without external sterilization. There was no contamination observed in the media tank throughout the process.

### Example 7: Production of Biomass by Fermentation of Methane Using pH Stat

About 5 liter Sartorius B plus automatic reactor filled with about 4 liter of growth media composition comprising about 0.1% of MgSO₄.7H₂O, about 0.02% of CaCl₂.2H₂O, about 0.0004% of Fe,Na-EDTA, about 0.00003% of NaMoO₄.2H₂O, about 0.00005% of FeSO₄.7H₂O, about 0.00004% of ZnSO₄.7H₂O, about 15 ppb of H₃BO₃, about 50 ppb of CoCl₂.6H₂O, about 0.25 ppm of Na₂-EDTA Dihydrate, about 20 ppb of MnCl₂.4H₂O, about 10 ppb of NiCl₂.6H₂O, about 1 ppm of CuSO₄.5H₂O, and 0.025% of nitrogen, was inoculated with M. capsulalus. The reactor run was initiated by continuously sparging about 0.1 1 pm of about 99.9% pure methane and about 0.06 1 pm of about 99.9% of oxygen at the bottom of the impeller. The input of growth media is gradually increased comprising about 0.4% of MgSO₄.7H₂O, about 0.1% of CaCl₂.2H₂O, about 0.0045% of Fe,Na-EDTA, about 0.0001% of NaMoO₄.2H₂O, about 0.0012% of FeSO₄.7H₂O, about 0.00015% of ZnSO₄.7H₂O, about 85 ppb of H₃BO₃, about 150 ppb of CoCl₂.6H₂O, about 2.5 ppm of Na₂-EDTA Dihydrate, about 50 ppb of MnCl₂.4H₂O, about 25 ppb of NiCl₂.6H₂O, about 7 ppm of CuSO₄.5H₂O about 0.5% of HNO3, about 0.5% of H2SO4, about 0.15% of H3PO4 and total nitrogen content of 0.25% was made by adding NaNO3 in the growth media, wherein the growth media composition is homogeneous and having pH of less than 3, preferably pH of about 2.5. The HNO3, H2SO4 and H3PO4 acted as acid to maintain pH, acid to dissolve all nutrients and nutrients for the growth of organism. This strategy of maintaining constant pH at 6.8 during the reaction with variation of 0.01 is called as pH stat. Eventual mass flow rates of carbon, oxygen, nitrogen, phosphate, sulphate, micro and trace elements were gradually increased in the ranges of 0.2-9 g/L/hour, 0.2-32 g/L/hour, 0.2-9 g/L/day, 0.1-4 g/L/day, 0.1-5 g/L/day, 0.3-17 g/L/day and 0.002-0.39 g/L/day, respectively, in order to support high specific growth rate and as well as their optimal concentrations in the reactor. Growth media was continuously fed to the reactor through Watson and Marlow peristaltic pump and fermentation broth was removed by Masterflex peristaltic pump.

Total solid biomass of about 2% was achieved in the broth which was removed at the rate of about 0.5 L/h.

In this method, pH was kept constant at about 6.8±0.4 by using acidic and basic compounds of nitrogen containing source, phosphate containing source and sulphate containing source along with diluted solutions of NaOH and HCl.

Reactor temperature was maintained at about 45±0.5° C. by use of thermo controller of the reactor unit.

The reactor was successfully run for the duration of about 600 hours with maintained productivity of 2.5 g/L/hour.

The growth media composition used for the production of biomass in this Example was as-prepared, without external sterilization. There was no contamination observed in the media tank throughout the process.

### Example 8: Enhanced Biomass Productivity by Fermentation of Methane and Nitrate as Nitrogen Source

About 5 liter of stirred tank reactor was filled with about 4 liter of growth media composition comprising about 0.1% of MgSO₄.7H₂O, about 0.02% of CaCl₂.2H₂O, about 0.0004% of Fe,Na-EDTA, about 0.00003% of NaMoO₄.2H₂O, about 0.00005% of FeSO₄.7H₂O, about 0.00004% of ZnSO₄.7H₂O, about 15 ppb of H₃BO₃, about 50 ppb of CoCl₂.6H₂O, about 0.25 ppm of Na₂-EDTA Dihydrate, about 20 ppb of MnCl₂.4H₂O, about 10 ppb of NiCl₂.6H₂O, about 1 ppm of CuSO₄.5H₂O, and about 0.025% of nitrogen in form of nitrate. The said growth media composition was inoculated with a starter culture of M. capsulalus. The reactor run was initiated with sparging about 0.1 1 pm of about 99.9% pure methane and 0.05 1 pm of about 99.9% pure oxygen at the bottom of the impeller. The reactor was maintained at about one atmospheric pressure. Once the cell density in the reactor reached about 0.15 to 2% biomass, the growth media composition comprising about 0.4% of MgSO₄.7H₂O, about 0.1% of CaCl₂.2H₂O, about 0.0045% of Fe,Na-EDTA, about 0.0001% of NaMoO₄.2H₂O, about 0.0012% of FeSO₄.7H₂O, about 0.00015% of ZnSO₄.7H₂O, about 85 ppb of H₃BO₃, about 150 ppb of CoCl₂.6H₂O, about 2.5 ppm of Na₂-EDTA Dihydrate, about 50 ppb of MnCl₂.4H₂O, about 25 ppb of NiCl₂.6H₂O, about 7 ppm of CuSO₄.5H₂O, about 0.3% of HNO₃, about 0.3% of H₂SO₄, and about 0.09% of H₃PO₄ was added to the reactor, wherein the growth media composition is homogeneous and having pH of less than 3, preferably pH of about 2.5. The total nitrogen content of 0.25% was made by adding NaNO₃ to the growth media composition. The dilution was gradually increased until productivity of about 4.5±0.1 g/l/hour was achieved in continuous steady state operation.

During the reaction, pH of about 6.8±0.3 was controlled by acidic and basic compounds of nitrogen containing source, phosphate sources and sulphate containing sources of the growth media composition, optionally along with NaOH/HCl. The reactor was run for a period of about 250 hours.

The growth media composition used for the production of biomass in this Example was as-prepared, without external sterilization. There was no contamination observed in the media tank throughout the process.

### Example 9: Enhanced Biomass Productivity by Fermentation of Methane and Ammonia as Nitrogen Source

About 5 liter of stirred tank reactor was filled with about 4 liter of growth media composition comprising about 0.1% of MgSO₄.7H₂O, about 0.02% of CaCl₂.2H₂O, about 0.0004% of Fe,Na-EDTA, about 0.00003% of NaMoO₄.2H₂O, about 0.00005% of FeSO₄.7H₂O, about 0.00004% of ZnSO₄.7H₂O, about 15 ppb of H₃BO₃, about 50 ppb of CoCl₂.6H₂O, about 0.25 ppm of Na₂-EDTA Dihydrate, about 20 ppb of MnCl₂.4H₂O, about 10 ppb of NiCl₂.6H₂O, about 1 ppm of CuSO₄.5H₂O, and about 0.025% of nitrogen in form of liquid ammonia, The said growth media composition was inoculated with a starter culture of M. capsulalus. The reactor run was initiated with sparging about 0.1 1 pm of about 99.9% pure methane and about 0.06 1 pm of about 90% pure oxygen at the bottom of the impeller. The reactor was maintained at about one atmospheric pressure. Once the cell density in the reactor reached about 0.1% biomass, the growth media composition comprising about 0.5% of MgSO₄.7H₂O, about 0.14% of CaCl₂.2H₂O, about 0.006% of Fe,Na-EDTA, about 0.00012% of NaMoO₄.2H₂O, about 0.0015% of FeSO₄.7H₂O, about 0.00019% of ZnSO₄.7H₂O, about 106 ppb of H₃BO₃, about 188 ppb of CoCl₂.6H₂O, about 3.1 ppm of Na₂-EDTA Dihydrate, about 63 ppb of MnCl₂.4H₂O, about 31 ppb of NiCl₂.6H₂O, about 8.6 ppm of CuSO₄.5H₂O, about 0.37% of H₂SO₄, and about 0.12% of H₃PO₄ was added to the reactor, wherein the growth media composition is homogeneous and having pH of less than 3, preferably pH of about 2.5. The liquid ammonia was added to reactor separately in order make nitrogen content of media to about 0.3%.

The dilution was gradually increased until productivity of 6±0.1 g/l hour was achieved in continuous steady state operation. During the reaction, pH was controlled by acidic and basic compounds of nitrogen containing source, phosphate sources and sulphate containing sources of the growth media composition, optionally along with NaOH/HCl. The reactor was run for a period of about 120 hours.

### Example 10: Scaled Up (10×) Production of Biomass by Fermentation of Methane

About 50 liter of stirred tank reactor (Biogenic Engineering Chennai) was filled with about 45 liter of growth media composition comprising about 0.1% of MgSO₄.7H₂O, about 0.02% of CaCl₂.2H₂O, about 0.0004% of Fe,Na-EDTA, about 0.00003% of NaMoO₄.2H₂O, about 0.00005% of FeSO₄.7H₂O, about 0.00004% of ZnSO₄.7H₂O, about 15 ppb of H₃BO₃, about 50 ppb of CoCl₂.6H₂O, about 0.25 ppm of Na₂-EDTA Dihydrate, about 20 ppb of MnCl₂.4H₂O, about 10 ppb of NiCl₂.6H₂O, about 1 ppm of CuSO₄.5H₂O, and 0.025% of nitrogen in form of liquid ammonia. The said growth media composition was inoculated with a starter culture of M. capsulalus. The reactor run was initiated with sparging about biogas containing about 76% methane and 99% pure oxygen at the bottom of the impeller. The reactor was maintained at about one atmospheric pressure. Once the cell density in the reactor reached about 0.25% biomass, the growth media composition comprising about 0.4% of MgSO₄.7H₂O, about 0.1% of CaCl₂.2H₂O, about 0.0045% of Fe,Na-EDTA, about 0.0001% of NaMoO₄.2H₂O, about 0.0012% of FeSO₄.7H₂O, about 0.00015% of ZnSO₄.7H₂O, about 85 ppb of H₃BO₃, about 150 ppb of CoCl₂.6H₂O, about 2.5 ppm of Na₂-EDTA Dihydrate, about 50 ppb of MnCl₂.4H₂O, about 25ppb of NiCl₂.6H₂O, about 7 ppm of CuSO₄.5H₂O, about 0.3% of H₂SO₄, and about 0.1% of H₃PO₄ was added to the reactor, wherein the growth media composition is homogeneous and having pH of less than 3, preferably pH of about 2.5. The liquid ammonia was added to reactor separately in order make nitrogen content of media to about 0.25%. Fermentation broth containing about 2 to 2.5% of solid biomass was continuously drawn out.

During the reaction, pH was controlled by acidic and basic compounds of nitrogen containing source, phosphate sources and sulphate containing sources of the growth media composition, optionally along with NaOH/HCl. The reactor was run for a period of about 120 hours.

The growth media composition used for the production of biomass in this Example was as-prepared, without external sterilization. There was no contamination observed in the media tank throughout the process.

1. A growth media composition comprising micro element and trace element, optionally along with nitrogen containing source, phosphate containing source and sulphate containing source.

2. The growth media composition as claimed in claim 1, wherein the microelement is selected from a group comprising MgSO₄.7H₂O, CaCl₂.2H₂O and a combination thereof; and
wherein the trace element is selected from a group comprising FeNa-EDTA, NaMoO₄.2H₂O, FeSO₄.7H₂O, ZnSO₄.7H₂O, H₃BO₃, CoCl₂.6H₂O, Na₂-EDTA Dihydrate, MnCl₂.4H₂O, NiCl₂.6H₂O, CuSO₄.5H₂O and a combination thereof.

3. (canceled)

4. The growth media composition as claimed in claim 1, wherein the growth media composition comprises MgSO₄.7H₂O, CaCl₂.2H₂O, Fe,Na-EDTA, NaMoO₄.2H₂O, FeSO₄.7H₂O, ZnSO₄.7H₂O, H₃BO₃, CoCl₂.6H₂O, Na₂-EDTA Dihydrate, MnCl₂.4H₂O, NiCl₂.6H₂O, CuSO₄.5H₂O, HNO₃, H₂SO₄/KHSO₄, H₃PO₄/KH₂PO₄, optionally along with additional nitrogen containing source, phosphate containing source, sulphate containing source or a combination thereof.

5. The growth media composition as claimed in claim 1, wherein the nitrogen containing source is selected from a group comprising sodium nitrate, sodium nitrite, potassium nitrate, potassium nitrite, ammonia, ammonium hydroxide, ammonium chloride, ammonium acetate, ammonium sulphate, nitric acid, di ammonium phosphate (DAP) and any combinations thereof; the phosphate containing source is selected from a group comprising potassium dihydrogen phosphate, dipotassium hydrogen phosphate, sodium dihydrogen phosphate, disodium hydrogen phosphate, phosphoric acid, di ammonium phosphate and any combinations thereof; and the sulphate containing source is selected from a group comprising copper sulphate, zinc sulphate, iron sulphate, magnesium sulphate, manganous sulphate, sulfuric acid and any combinations thereof.

6. The growth media composition as claimed in claim 1, wherein the growth media composition comprises MgSO₄.7H₂O in an amount ranging from about 0.1% to 1.2%, CaCl₂.2H₂O in an amount ranging from about 0.02% to 0.3%, FeNa-EDTA in an amount ranging from about 0 to 0.013%, NaMoO₄.2H₂O in an amount ranging from about 0.000026% to 0.0003%, FeSO₄.7H₂O in an amount ranging from about 0.00005% to 0.006%, ZnSO₄.7H₂O in an amount ranging from about 0.00004% to 0.0005%, H₃BO₃ in an amount ranging from about 0 ppm to 0.3 ppm, CoCl₂.6H₂O in an amount ranging from about 0.05 ppm to 0.45 ppm, Na₂-EDTA Dihydrate in an amount ranging from about 0 ppm to 8 ppm, MnCl₂.4H₂O in an amount ranging from about 0.02 ppm to 0.15 ppm, NiCl₂.6H₂O in an amount ranging from about 0.01 ppm to 0.075 ppm, CuSO₄.5H₂O in an amount ranging from about 1 ppm to 50 ppm.

7. The growth media composition as claimed claim 1, wherein ratio of elemental nitrogen to phosphate is ranging from about 1:2 to 10:1; ratio of elemental nitrogen to sulphate is ranging from about 1:1 to 10:1; and
wherein the growth media composition is having pH of less than 3, preferably pH of about 2.5; and the growth media composition is homogenous and self-sterilized.

8. (canceled)

9. A process of preparing the growth media composition as claimed in claim 1, comprising mixing micro element and trace element, optionally along with nitrogen containing source, phosphate containing source and sulphate containing source by a predetermined technique at a predetermined temperature for a predetermined duration to obtain the growth media composition.

10. A method of producing biomass comprising—
culturing microorganism in a growth media composition;
supplementing the culture with a growth media composition having acidic pH; and
harvesting the biomass.

11. The method as claimed in claim 10, wherein the culturing of the microorganism comprises—
inoculating the growth media composition with the microorganism, followed by providing gaseous substrate and oxygen; and
monitoring cell density of the microorganism and supplementing with the growth media composition.

12. The method as claimed in any one of claim 10, wherein the growth media composition for culturing and supplementing is same or different; and wherein the growth media composition for supplementing the culture is having a pH of less than 3, preferably pH of about 2.5;
wherein the culturing of the microorganism is carried out at a temperature ranging from about 5° C. to 50° C., at a pressure ranging from about 0 bar to 5 bar, for a duration ranging from about 120 hours to 900 hours.

13. (canceled)

14. The method as claimed in claim 11, wherein the growth media composition is supplemented when the cell density of the microorganism is ranging from about 0.15% to 2%.

15. The method as claimed in claim 11, wherein the gaseous substrate is selected from a group comprising methane, natural gas, syngas, landfill gas, carbon monoxide, biogas and any combinations thereof; and the gaseous substrate is at a concentration ranging from about 1 mg/L to 8 mg/L; and the oxygen is at a concentration ranging from about 1 mg/L to 10 mg/L; and
wherein the microorganism is selected from a group comprising Methylococcus capsulatus, Methylobacterium extorquens, Methylomicrobium album, Methylocapsa acidiphila, Methylobacterium organophilum, Methylobacterium mesophilicum, Methylobacterium dichloromethanicum, Methylocella silvestris, Methylosinus trichosporium, Methylobacillus flagellatus KT, Methylibium petroleiphilum PM1, Methylobacterium nodulans, Methylobacterium populi, Methylobacterium chloromethanicum, Methylacidiphilum infernorum V4, Methylophilus methylotrophus, Methylomonas methanica, Methylobacterium rhodesianum MB 126, Methylobacter tundripaludum, Methylobacterium sp. 4-46, Methylovorus glucosetrophus SIP3-4, Mycobacterium smegmatis, Methylobacterium rhodesianum, Methylosinus sporium, Methylocella palustris, Methylobacterium fujisawaense, Methylocystis parvus, Methylovulum miyakonense, Methylobacterium rhodinum, Methylocystis echinoides, Methylomonas rubra, Methylococcus thermophilus, Methylobacterium aminovorans, Methylobacterium thiocyanatum, Methylobacterium zatmanii, Acidithiobacillus ferrivorans, Methylobacterium aquaticum, Methylobacterium suomiense, Methylobacterium adhaesivum, Methylobacterium podarium, Methylobacter whittenburyi, Crenothrix polyspora, Clonothrix fusca, Methylobacter bovis, Methylomonas aurantiaca, Methylomonas fodinarum, Methylobacterium variabile, Methylocystis minimus, Methylobacter vinelandii, Methylobacterium hispanicum, Methylomicrobium japanense, Methylococcaceae bacterium, Methylocystis methanolicus and any combination thereof.

16. (canceled)

17. The method as claimed in claim 10, wherein the biomass is produced at an enhanced productivity rate ranging from about 0.5 g L⁻¹ hour⁻¹ to 8 g L⁻¹ hour⁻¹.

18. A method of producing value added products comprising:
culturing microorganism in a growth media composition;
supplementing the culture with a growth media composition having acidic pH;
harvesting biomass; and
separating therefrom value added products from the biomass

19. The method as claimed in claim 18, wherein the culturing of the microorganism comprises—
inoculating the growth media composition with the microorganism, followed by providing gaseous substrate and oxygen; and
monitoring cell density of the microorganism and supplementing with the growth media composition.

20. The method as claimed in claim 18, wherein the growth media composition for culturing and supplementing is same or different; and wherein the growth media composition for supplementing the culture is having pH of less than 3, preferably pH of about 2.5.

21. The method as claimed in claim 19, wherein the growth media composition is supplemented when the cell density of the microorganism is ranging from about 0.15% to 2%; the gaseous substrate is selected from a group comprising methane, natural gas, syngas, landfill gas, carbon monoxide, biogas and any combinations thereof; and the gaseous substrate is at a concentration ranging from about 1 mg/L to 8 mg/L; and the oxygen is at a concentration ranging from about 1 mg/L to 10 mg/L.

22. The method as claimed in claim 18, wherein the culturing of the microorganism is carried out at a temperature ranging from about 20° C. to 50° C., at a pressure ranging from about 0 bar to 5 bar, for a duration ranging from about 120 hours to 900 hours; and
wherein the microorganism is selected from a group comprising Methylococcus capsulatus, Methylobacterium extorquens, Methylomicrobium album, Methylocapsa acidiphila, Methylobacterium organophilum, Methylobacterium mesophilicum, Methylobacterium dichloromethanicum, Methylocella silvestris, Methylosinus trichosporium, Methylobacillus flagellatus KT, Methylibium petroleiphilum PM1, Methylobacterium nodulans, Methylobacterium populi, Methylobacterium chloromethanicum, Methylacidiphilum infernorum V4, Methylophilus methylotrophus, Methylomonas methanica, Methylobacterium rhodesianum MB 126, Methylobacter tundripaludum, Methylobacterium sp. 4-46, Methylovorus glucosetrophus SIP3-4, Mycobacterium smegmatis, Methylobacterium rhodesianum, Methylosinus sporium, Methylocella palustris, Methylobacterium fujisawaense, Methylocystis parvus, Methylovulum miyakonense, Methylobacterium rhodinum, Methylocvstis echinoides, Methylomonas rubra, Methylococcus thermophilus, Methylobacterium aminovorans, Methylobacterium thiocyanatum, Methylobacterium zatmanii, Acidithiobacillus ferrivorans, Methylobacterium aquaticum, Methylobacterium suomiense, Methylobacterium adhaesivum, Methylobacterium podarium, Methylobacter whittenburyi, Crenothrix polyspora, Clonothrix fusca, Methylobacter bovis, Methylomonas aurantiaca, Methylomonas fodinarum, Methylobacterium variabile, Methylocystis minimus, Methylobacter vinelandii, Methylobacterium hispanicum, Methylomicrobium japanense, Methylococcaceae bacterium, Methylocvstis methanolicus and any combination thereof.

23. (canceled)

24. (canceled)

25. The method as claimed in claim 18, wherein the value added product is selected from a group comprising lactic acid, succinic acid, formic acid, acetic acid, malic acid, beta-carotene, lutein, zeaxanthin, lycopene, astaxanthin, methanobactin, ectoine, indigo, peptide, mandelic acid and annatto.

26. The method as claimed in claim 25, wherein the lactic acid is produced at a concentration ranging from about 5 g/l to 120 g/l, the succinic acid is produced at a concentration ranging from about 5 g/l to 50 g/l, the formic acid is produced at a concentration ranging from about 5 g/l to 50 g/l, the acetic acid is produced at a concentration ranging from about 5 g/l to 50 g/l, the malic acid is produced at a concentration ranging from about 5 g/l to 50 g/l, the beta-carotene is produced at a concentration ranging from about 0.5 g/l to 50 g/l, the lutein, the zeaxanthin, the lycopene and the annatto is produced at concentration ranging from about 0.5 g/l to 5 g/l, respectively and the methanobactin is produced at a concentration ranging from about 1 g/l to 5 g/l.
